(19) United States
(12) Patent Application Publication
(10) Pub. No.: US 2010/0256502 A1
(43) Pub. Date: Oct. 7, 2010

(54) MATERIALS AND PROCESSES FOR BONDING ACOUSTICALLY NEUTRAL STRUCTURES FOR USE IN ULTRASOUND CATHETERS

(75) Inventors: Donald Joseph Buckley, Schenectady, NY (US); Douglas Glenn Wildes, Ballston Lake, NY (US); Warren Lee, Niskayuna, NY (US); Weston Blaine Griffin, Niskayuna, NY (US)

Correspondence Address: GENERAL ELECTRIC COMPANY;GLOBAL RESEARCH, ONE RESEARCH CIRCLE, BLDG. K1-3A59, NISKAYUNA, NY, 12309, US

(73) Assignee: General Electric Company, Schenectady, NY (US)

(21) Appl. No.: 12/418,824

(22) Filed: Apr. 6, 2009

(51) Int. Cl. A61B 8/14; B29C 45/14

(52) U.S. Cl. 600/466; 264/272.16

(57) ABSTRACT

Provided herein is an ultrasound probe comprising a probe housing defining a distal end, an ultrasonic transducer array disposed within the probe housing and rotatable within said probe housing, an acoustically neutral structure bonded to a surface of the ultrasonic transducer array by an adhesive, a motor coupled to the ultrasonic transducer array, the motor being configured to rotate the ultrasonic transducer array in order to image a three-dimensional volume; and an acoustic coupling fluid disposed within free volume of the probe housing. Also provided is a method of manufacturing.

### BACKGROUND OF THE INVENTION

Catheter-based ultrasound imaging techniques are interventional procedures that generally involve inserting a probe, such as an imaging catheter, into a vein, such as the femoral vein, or an artery. The probes are specially designed to provide two-dimensional or real-time three-dimensional imaging. Such applications are demanding and may require very small transducer packages that are nevertheless capable of collecting large amount of information. In some circumstances, it may be desirable to provide some form of acoustic coupling between the transducer assembly and the surrounding ultrasound probe housing to provide an effective or suitable acoustic transition between the transducer and the housing

The presence of acoustic fluid however, may degrade image quality if bubbles form in the fluid, due to mechanical rotation of the transducer, and the bubbles interfere with the imaging. The acoustic coupling fluid may also cause undesirable focusing effects if the sound velocity in the coupling fluid is different than the sound velocity in the imaged medium (i.e., blood or tissue). Therefore a need exists for the development of a method to minimize the risk of bubble formation and interference of the acoustic fluid. One approach may be to design an ultrasound probe using acoustically neutral material to occupy the space between the transducer and the probe housing.

### BRIEF DESCRIPTION OF THE INVENTION

This invention describes materials and processes for manufacture of an acoustically neutral material that may be used in an ultrasound probe housing. Choice of material, based on acoustic properties, and process to manufacture a part, which is readily mountable to other components of an ultrasound probe, is described.

In one embodiment, the present invention provides an ultrasound probe comprising a probe housing defining a distal end, an ultrasonic transducer array disposed within the probe housing and rotatable within said probe housing, an acoustically neutral structure bonded to a surface of the ultrasonic transducer array by an adhesive, a motor coupled to the ultrasonic transducer array, said motor being configured to rotate the ultrasonic transducer array in order to image a three-dimensional volume; and an acoustic coupling fluid disposed within free volume of the probe housing.

In another embodiment, the present invention provides a method of manufacturing an ultrasound probe. The ultrasound probe comprises a probe housing defining a distal end, an ultrasonic transducer array disposed within the probe housing and rotatable within the probe housing, an acoustically neutral structure bonded to a surface of the ultrasonic transducer array by an adhesive and wherein the acoustically neutral structure comprises a polymer cap bonded to a polymer film base. The said method of manufacturing comprises molding the polymer cap to the polymer film base using injection molding, compression molding, or a combination thereof.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of a mold assembly for a two layered acoustically neutral structure for use in combination with an injection-molding device.

FIG. 2 is a flow chart outlining process steps for molding the two layered acoustically neutral material in accordance with an embodiment of the invention

FIG. 3 is a partially cutaway schematic illustration of an intracardiac echocardiography (ICE) catheter for use with an embodiment of the invention.

FIG. 4 is a cross-section illustration of the ICE catheter shown in FIG. 3.

### DETAILED DESCRIPTION OF THE INVENTION

This invention describes materials and processes for manufacture of an acoustically neutral structure, for use at ultrasonic frequencies in an ultrasound probe. Ultrasound probes include, but are not limited to, endoscopes, intraoperative or intracavity ultrasound probes, and ultrasound catheters. Ultrasound catheters, which may incorporate an embodiment of the invention include, but are not limited to, transesophageal catheters, transnasal catheters, transthoracic catheters, intracavity catheters, intracardiac catheters, intravascular catheters, and intraoperative catheters.

An ultrasound probe may be configured to image a three-dimensional volume and comprise a probe housing having a transducer array disposed within the housing, and a motor coupled to the transducer array. The motor is configured to rotate the transducer array in order to image a three-dimensional volume. Free space within the ultrasound probe housing may be filled with an acoustic coupling fluid such as water, propylene glycol, saline, mineral oil, ethylene glycol, castor oil, or a combination thereof. Typically the coupling fluid would have acoustic impedance and sound velocity near those of the imaging medium such as blood and tissue (Z˜1.5 M Rayl, V˜1540 m/sec). While the coupling fluid assists in imaging, a problem with the acoustic fluid is the tendency of the fluid to form bubbles during operation of the probe. The bubbles may form due to incomplete filling of the probe housing leaving air voids in the chamber and may also form during operation of the motor. The bubbles may interfere with image acquisition if they are within the acoustic path. Reducing the amount of free volume in the probe housing and the spacing between the housing and the transducer may alleviate image quality degradation due to the presence of bubbles in the acoustic path. A solid material may be used as a filler.

Transmission of ultrasound through a material may result in modification of the transmitted beam profile and reflection or absorption of energy. This also applies to a solid material used in a probe housing as a filler. An acoustic impedance mismatch between an ultrasound probe component material and another material may cause reflection of energy at the material interfaces and lead to reverberation and a loss of axial resolution in an image. If the acoustic path length through the probe housing material is not uniform, then an acoustic velocity mismatch between the material and adjacent components may cause a lens effect, which can focus, de-focus, or distort the ultrasound beam, substantially reducing resolution and contrast in an image. Therefore a material which is acoustically neutral would be the preferred as filler. Acoustically neutral materials for use in the application include, but are not limited to, thermoplastic elastomers, polyurethanes, polymethylpentene, low density polyethylene, ethylene vinyl acetate (EVA), and filled silicones. One example of a thermoplastic elastomer is a polyether-amide block copolymer.

In certain embodiments the acoustically neutral material may comprise a two-layered structure such as a polymer cap bonded to a polymer film base. In one embodiment, a polyether-amide block copolymer having a controllable ratio of soft to hard blocks may be used as a polymer cap. By varying the molecular weight of the relatively low modulus, soft ether block relative to that of relatively high modulus, hard amide block, the elastic modulus of the copolymer and the acoustic properties dependent on modulus can be varied, more or less continuously, across a wide range. This degree of freedom allows selection of an ether-to-amide block ratio such that the material has sound velocity and acoustic impedance similar to those of water, tissue, or acoustically equivalent coupling fluids. The result is an acoustically neutral material relative to water, tissue or coupling fluids, which is transparent to the ultrasonic radiation and may have minimal effect on an ultrasonic beam passing through other than reduction of transmitted intensity by absorption. An example of such a material series are the PEBAX resins offered by Arkema, of which PEBAX 2533 grade is especially suitable.

However such materials, being both elastomeric and partially amide-based, may be difficult to bond to other components of the ultrasound system. The amide block imparts crystallinity and a consequent degree of chemical resistance to the composite, so that it does not readily enter into bond-forming reactions with commonly used adhesives, such as epoxies, acrylates, or silicones.

In one embodiment, a low speed injection-compression process in which the block copolymer is molded against a polymer film base, the polymer film base having better adhesion facilitates bonding. An example of a polymer film base with better adhesion is a polyimide such as polyimide Kapton film available from Dupont. Bonding between the copolymer and the polyimide film may occur through interfacial adhesion. Interfacial adhesion results from molding the copolymer to the polyimide using injection molding, compression molding, or a combination thereof. The resulting two-layered copolymer/film composite, which is used as the acoustically neutral structure, may then be bonded to adjacent components using conventional techniques.

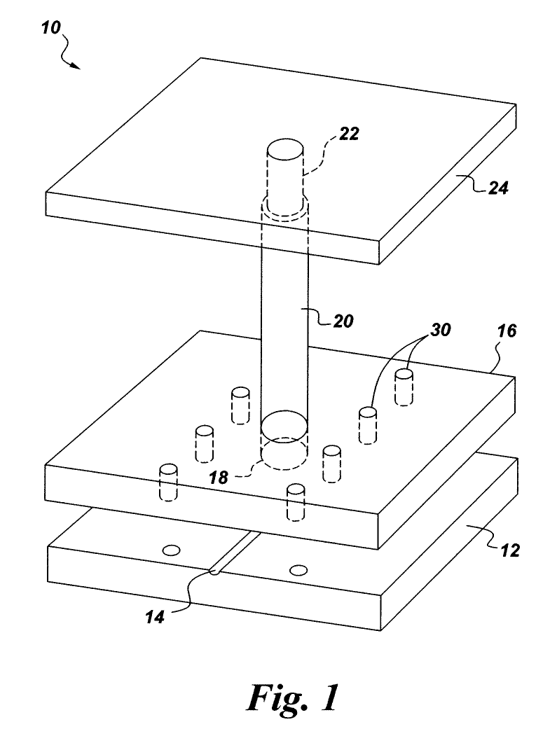

Referring to FIG. 1 an injection/compression mold 10 consists of a bottom plate 12 with a cylindrical groove 14, a plane top plate 16 with a center hole 18, an injection column 20 attached to and feeding through the center hole in the top plate, an injection ram 22 which slides snugly in the bore of the injection column, and a pressure plate 24 attached to the top of the injection ram. The bottom and top mold plates 12 and 16 are secured to each other by machine screws 30, placed between the platens of a programmable compression molding press, not shown, and brought to temperature. This sub-assembly is removed from the press, and the injection column 20, whose bottom end has been filled with tightly packed disks of the copolymer, is quickly screwed into the top plate, and the pressure plate attached. The complete assembly is placed between the molding press platens, brought to temperature and lightly compressed until polymer flows from the exit holes.

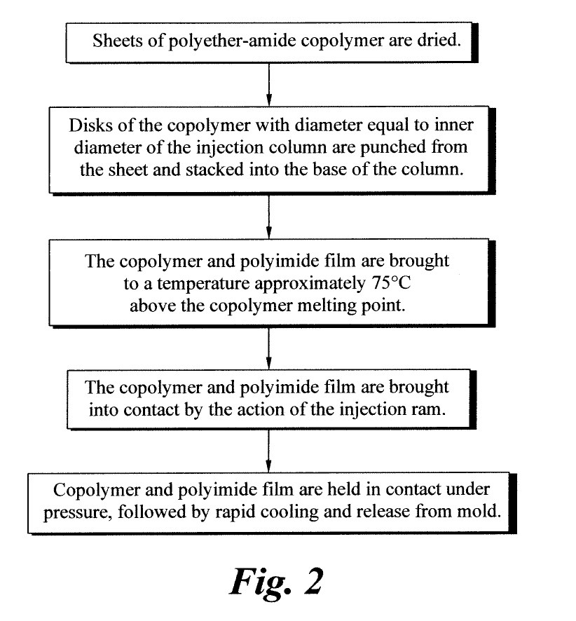

One embodiment of a molding process is illustrated in the flow diagram of FIG. 2 and defined by the following general steps using a polyether-amide copolymer cap and a polyimide film base: 1) sheets of polyether-amide copolymers are dried, 2) disks of the copolymer with diameter equal to the inner diameter of the injection column are punched from the sheet and stacked in to the base of the column, 3) the copolymer and polyimide film are brought to a temperature approximately 75 degree C. above the copolymer melting point, 4) the copolymer and polyimide film are brought into contact by the action of the injection ram, and 5) the copolymer and polyimide film are held in contact under pressure, followed by rapid cooling and release from the mold.

Referring again to FIG. 1, in certain embodiments, the sheets of polyether-amide copolymer may be dried in vacuum at 60 degrees C. for a minimum of 48 hours prior to molding. The disks of the copolymer with diameter equal to inner diameter of the injection column are punched from sheet and stacked tightly into the base of column 20 to minimize air entrapment. The column containing the copolymer disks is screwed into top plate 16 of the mold subassembly, which may be preheated to a temperature approximately 75 degrees C. above the melting point of the copolymer. The complete mold assembly 10 may be placed between the platens of a compression molding machine and the mold assembly may be brought again to a temperature approximately 75 degrees C. above the melting point of the copolymer. The lower platen of the press may be slowly raised, under minimal pressure, thereby forcing the injection ram 22 down into the column, until polymer is ejected from relief holes at either end of the cylindrical channel 14 in the bottom plate 12. The pressure may be increased to 9000 psi for one minute to insure consolidation of the part and the mold may then be rapidly cooled in place still under 9000 psi pressure to room temperature after which time the platens may be opened and the assembly removed from the press. The mold may then be disassembled and the composite polyimide film/copolymer part removed from the mold.

The polyimide film assists demolding as well as serving as an attachable base for the copolymer. The combination of extended drying in vacuum and exposure to temperature well above the melting point of the copolymer during molding serves to eliminate or reduce contaminants in the copolymer, which may otherwise inhibit adhesion.

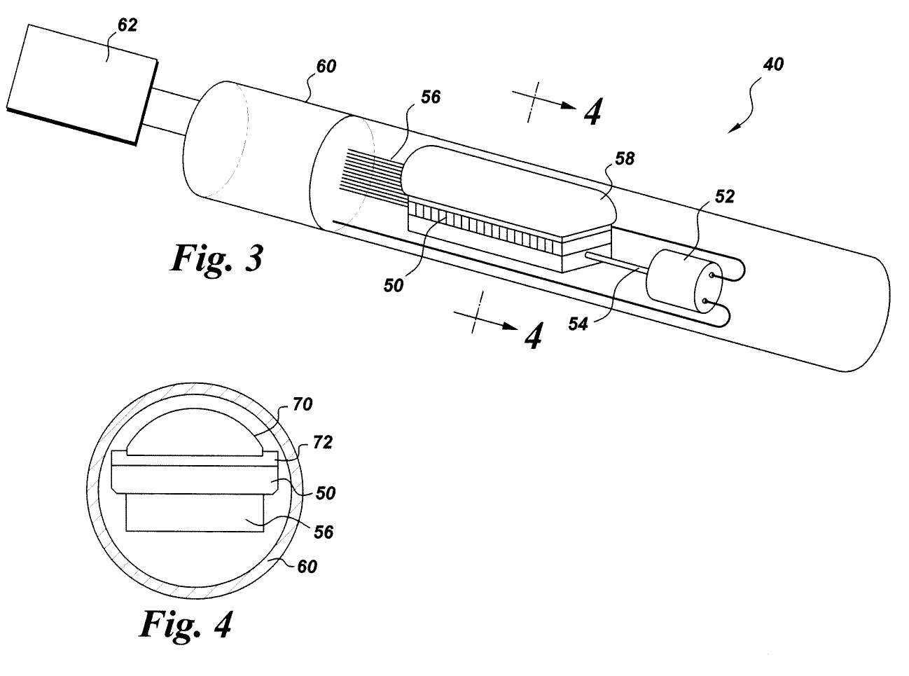

Referring to FIG. 3, an illustration of an intracardiac echocardiography (ICE) catheter 40 is shown which may incorporate the acoustically neutral structures described above. It should be appreciated that the ICE catheter 40 is described for illustrative purposes, and that any ultrasound probe adapted to transmit or receive ultrasonic frequencies may alternatively be implemented in place of the ICE catheter 40. Ultrasound probes include, but are not limited to, endoscopes, intraoperative or intracavity ultrasound probes, and ultrasound catheters. Ultrasound catheters, which may incorporate an embodiment of the invention, include but are not limited to transesophageal catheters, transnasal catheters, transthoracic catheters, intracavity catheters, intracardiac catheters, intravascular catheters, and intraoperative catheters.

The ICE catheter 40 shown in FIG. 3, comprises a transducer array 50, a motor 52, which may be internal or external to the space-critical environment, a drive shaft 54 or other mechanical connections between motor 52 and the transducer array 50, and an interconnect 56. The ICE catheter 40 further includes a catheter housing 60 enclosing the transducer array 50, motor 52, interconnect 56 and drive shaft 54. The acoustically neutral structure 58 is bonded to the transducer using an adhesive. The acoustically neutral structure 58 is designed to reduce free volume within the catheter housing while not interfering with the operation of the transducer array or motor. Specifically, the free volume between the transducer and the catheter housing is reduced due to the presence of the acoustically neutral structure. The small curved, space remaining between the acoustically neutral structure and the catheter housing promotes filling with the acoustic coupling fluid by capillary action. In one embodiment, the structure 58 is cylindrical. In other embodiments, the structure 58 is a right circular cylinder whose lateral surface contains segments that are perpendicular to the base. In still other embodiments the structure 58 parallels the catheter housing. The distance between the surface of the catheter housing 60 facing the acoustically neutral structure and the acoustically neutral structure 58 depends on the catheter design. In one embodiment the distance may be less than 3 mils.

As shown in the depicted embodiment in FIG. 3, the transducer array 50 is mounted on drive shaft 54 and the transducer array 50 is rotatable with the drive shaft 54. Motor controller 62 and motor 52 control the rotational motion of the transducer array 50. Interconnect 56 refers to, for example, cables and other connections coupling the transducer array 50 with an ultrasound imaging device (not shown) for use in receiving and/or transmitting signals.

The catheter housing 60, or at least the portion that intersects the ultrasound imaging volume, is acoustically transparent, e.g. low attenuation and scattering, acoustic impedance near that of blood and tissue. The space between the transducer and the housing may be filled with an acoustic coupling fluid (not shown), e.g., water, also with acoustic impedance and sound velocity near those of blood and tissue (Z equal to approximately 1.5 M Rayl, V equal to approximately 1540 m/sec). In one embodiment, the acoustically neutral material may have a sound velocity in the range 1.0 to 3.0 millimeters per microsecond, and acoustic impedance in the range of 1.0 to 3.0 MegaRayls (MRayls).

An additional advantage of incorporating an acoustically neutral solid filler material between the transducer and the catheter housing is that the shape of the filler material can be specifically designed to conform to the inside of the catheter housing, minus a small uniform gap. This has the effect of somewhat relaxing the sound velocity requirement on the acoustic coupling fluid. Since the coupling fluid would only occupy the small uniform gap between the solid filler material and the catheter housing, detrimental focusing effects due to a mismatched sound velocity of the coupling fluid will be minimized.

A cross section of the ICE catheter 40 depicted in FIG. 3 is shown in FIG. 4. The dimensions of the individual components may vary based on the specific application. The acoustically neutral structure 58, composed of a polyether-polyamide copolymer cap 70 and a polyimide base 72, is bonded to the surface of the ultrasonic transducer 50. The catheter housing 60 is shown as well as the interconnect 56. Dimensions of one embodiment of the invention may vary based on the application. In certain embodiments, the radius of the catheter may be between 0.5 and 2.0 mm.

The invention may be embodied in other specific forms without departing from the spirit or essential characteristics thereof. The foregoing embodiments are therefore to be considered in all respects as illustrative rather than limiting on the invention described herein. The scope of the invention is thus indicated by the appended claims rather than by the foregoing description, and all changes that come within the meaning and range of equivalency of the claims are therefore intended to be embraced therein.

We claim:

1. An ultrasound probe comprising:
a probe housing defining a distal end;
an ultrasonic transducer array disposed within the probe housing and rotatable within said probe housing;
an acoustically neutral structure bonded to a surface of the ultrasonic transducer array by an adhesive;
a motor coupled to the ultrasonic transducer array, said motor being configured to rotate the ultrasonic transducer array in order to image a three-dimensional volume; and
an acoustic coupling fluid disposed within free volume of the probe housing.

2. The ultrasound probe of claim 1, wherein the ultrasound probe is an ultrasound catheter, an endo scope, an intraoperative ultrasound probe, an intracavity ultrasound probe, an ultrasound imaging probe or an ultrasound therapy device.

3. The ultrasound probe of claim 2 wherein the ultrasound catheter is a transesophageal catheter, an intra-cardiac echocardiographic catheter, a transnasal catheter, a transthoracic catheter, an intravascular catheter, an intracavity catheter, or an intraoperative catheter.

4. The ultrasound probe of claim 3 wherein the ultrasound catheter is an intra-cardiac echocardiographic catheter.

5. The ultrasound probe of claim 1, wherein the acoustic coupling fluid comprises water, propylene glycol, saline, mineral oil, ethylene glycol, castor oil, or a combination thereof.

6. The ultrasound probe of claim 1, wherein the acoustically neutral structure comprises a two-layered structure.

7. The ultrasound probe of claim 6, wherein the two-layer structure comprises a polymer cap bonded to a polymer film base.

8. The ultrasound probe of claim 7 wherein the polymer cap comprises a polyether-polyamide block copolymer and the polymer film comprises a polyimide.

9. The ultrasound probe of claim 6, wherein the two-layer structure is bonded together through interfacial adhesion.

10. The ultrasound probe of claim 1, wherein the acoustically neutral structure is bonded to the transducer array by a silicone adhesive.

11 The ultrasound probe of claim 1 wherein the acoustically neutral structure has a right circular cylindrical geometry.

12. The ultrasound probe of claim 1 wherein the distance between a surface of the probe housing facing the acoustically neutral structure and the acoustically neutral structure is less than 10 mils.

13. The ultrasound probe of claim 1 wherein the acoustically neutral structure has a sound velocity in the range 1.0 to 3.0 millimeters per microsecond, and acoustic impedance in the range of 1.0 to 3.0 MegaRayls.

14. A method of manufacturing an ultrasound probe comprising:
a probe housing defining a distal end;
an ultrasonic transducer array disposed within the probe housing and rotatable within said probe housing;
an acoustically neutral structure bonded to a surface of the ultrasonic transducer array by an adhesive said acoustically neutral structure comprising a polymer cap bonded to a polymer film base; and
wherein said method comprises molding the polymer cap to the polymer film base using injection molding, compression molding, or a combination thereof.

15. The method of manufacturing an ultrasound probe according to claim 14 wherein the polymer cap comprises a polyether-polyamide block copolymer and the polymer film base comprises a polyimide.

16. The method of manufacturing an ultrasound probe according to claim 14 further comprising the step of bonding the acoustically neutral structure to the ultrasonic transducer array by applying a silicone adhesive between a surface of the acoustically neutral structure and the surface of the ultrasonic transducer.
